(19) United States
(12) Patent Application Publication
(10) Pub. No.: US 2020/0005892 A1
(43) Pub. Date: Jan. 2, 2020

(54) CONTENT MANAGEMENT SYSTEM FOR CREATION OF LIVING LAB REPORTS

(71) Applicant: MY GENE COUNSEL, LLC, Branford, CT (US)

(72) Inventors: Ellen T. MATLOFF, Branford, CT (US); Danielle C. BONADIES, Branford, CT (US)

(21) Appl. No.: 16/455,376

(22) Filed: Jun. 27, 2019

Related U.S. Application Data
(60) Provisional application No. 62/692,214, filed on Jun. 29, 2018.

(51) Int. Cl. G16B 20/00; G16H 10/40; G16B 50/00

(52) U.S. Cl. CPC G16B 20/00; G16B 50/00; G16H 10/40

(57) ABSTRACT

The present disclosure provides a method for analyzing and providing insight and advice on genetic test results in the form of Living Lab Reports (LLRs). LLRs can be created within a web-based content management system (CMS) through a Data Definition Model (DDM). The DDM can determine the hierarchical order and structure of the LLR. The DDM can link the LLRs created within the CMS to users' genetic testing lab reports within a direct web portal. These LLRs can then be viewed by end users using a direct web portal.

### CROSS-REFERENCE

This application claims the benefit of U.S. Provisional Patent Application No. 62/692,214, filed Jun. 29, 2018, the contents of which is incorporated herein by reference in its entirety.

### BACKGROUND

Genetics is a rapidly changing field, involving thousands of genes. As many as 60% of individuals are candidates for genetic testing. Early discovery and monitoring of diseases is beneficial in a number of healthcare settings. In some cases, disease development may be delayed or prevented entirely based on genetic test results. Healthcare providers from many different clinical backgrounds order and interpret these test results. However, the rate of misinterpretation of test results will likely continue to increase as genetic testing becomes more widely available and complex. Genetic test reports are stagnant, meaning new, clinically-relevant findings are often never reported to clinicians or patients. Such challenges threaten the potential of genomics and personalized medicine.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some embodiments, the disclosure provides a computer program product comprising a non-transitory computer-readable medium having computer-executable code encoded therein, the computer-executable code adapted to be executed to implement a method comprising: a) providing a content management system, wherein the content management system comprises: I) an information input module; II) a report assignment module; III) a review module; IV) an output module; and V) a report update module; b) receiving, by the information input module, information obtained from a genetic analysis of a biological sample from a subject; c) performing an assignment analysis, by the report assignment module, of the information obtained from the genetic analysis; d) generating, by the report assignment module, a tentative assignment of a report to the subject based on the assignment analysis, wherein the report comprises an informational text that pertains to the information obtained from the genetic analysis; e) storing, by the review module, the tentative assignment, wherein the tentative assignment remains stored until the tentative assignment is reviewed and approved by an administrator; f) generating by the review module, based on receipt of approval by the administrator, an assigned report comprising the informational text; g) transmitting the assigned report from the review module to the output module; h) displaying by the output module the assigned report to a user; i) updating, by the report update module, the informational text, thereby generating an updated report, wherein the updated report is generated after a new piece of information is received by the report update module; and j) transmitting the updated report from the report update module to the output module, wherein the output module displays the updated report to the user.

### BRIEF DESCRIPTION OF THE DRAWINGS

FIG. 1 shows a process for editing a question and answer database and granting permission rights.

FIG. 2 shows a process for editing Living Lab Reports (LLRs) and granting permission rights.

FIG. 3 illustrates an example push notification alerting a user to the LLR update.

FIG. 4 shows a non-limiting example of a computer control system that is programmed or otherwise configured to implement methods provided herein.

FIG. 5 shows an interactive browser displaying an LLR view on the web portal.

FIG. 6 illustrates an interactive browser expanding the overview tab of the LLR assigned to a user who tested positive with a pathogenic variant or mutation in BRCA1.

FIG. 7 shows the screen view of FIG. 6 once the user expands one of the listed questions.

FIG. 8 shows a lab integration data flow diagram.

### DETAILED DESCRIPTION

The disclosure provides a method for analyzing and providing insight and advice on genetic analyses (e.g. genetic test results) in the form of Living Lab Reports (LLRs). In some embodiments, LLRs comprise informational text pertaining to information obtained from a genetic analysis. LLRs can be created within a web-based content management system (CMS) through a unique “Data Definition Model” (DDM). The DDM can determine the hierarchical order and structure of the LLR. The DDM can be used to link the LLRs created within the CMS to users' genetic testing lab reports within a report assignment module. These LLRs can then be viewed by end users using the output module. The DDM can link individual test results to genetic expert content, provide actionable genomic insights that allow clinicians to utilize genomic analyses in clinical care, decrease errors in misinterpretation and patient management, allow consumers to utilize genomic information, send notifications when content updates, and deliver digital genetic counseling.

The genetic analyses can be obtained from processing a biological sample or sequencing the biological sample without any involvement from the user. The profiles of at least one or more markers of a disease or condition can be compared. This comparison can be quantitative or qualitative. Quantitative measurements can be taken using any of the assays described herein. Assaying can comprise processing a biological sample and/or sequencing of the biological sample without any involvement from a user. Non-limiting examples of assays include, sequencing, direct sequencing, random shotgun sequencing, Sanger dideoxy termination sequencing, whole-genome sequencing, exome sequencing, transcriptome sequencing, cell-free DNA sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD sequencing, MS-PET sequencing, mass spectrometry, matrix assisted laser desorption/ionization-time of flight (MALDI-TOF) mass spectrometry, electrospray ionization (ESI) mass spectrometry, surface-enhanced laser desorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), polymerase chain reaction (PCR) analysis, quantitative PCR, real-time PCR, fluorescence assay, colorimetric assay, chemiluminescent assay, or a combination thereof. The sequencing can be, for example, whole genome sequencing, low pass whole genome sequencing, or targeted sequencing. The sequencing can be, for example, whole transcriptome sequencing on RNA, such as tumor RNA.

In addition to sequencing, other reactions and/operations can occur during preparation of the genetic analyses, including as non-limiting examples: nucleic acid quantification, sequencing optimization, detecting gene expression, quantifying gene expression, genomic profiling, cancer profiling, and analysis of expressed markers. Other example forms of assaying include immunohistochemistry profiling and genomic profiling of the biological sample. Using immunohistochemistry, antigens can be identified during examination of the tumor and normal tissue cells of the biological sample. Immunohistochemistry can also provide results on the distribution and localization of biomarkers and differentially expressed proteins in different locations of the biological sample tissue, for example, over- or under-expressed proteins.

In some embodiments, a genetic analysis comprises using genome profiling following sequencing to determine and measure the activity of thousands of genes simultaneously. The profiling can distinguish between cells that are actively dividing. Genomic profiling measures how well cells respond to a particular treatment. The patterns in the tumor DNA are determined, for example, by comparing the tumor DNA against a set of known DNA. The group of genes whose combined expression pattern is uniquely characteristic to a given condition establishes the gene signature of the particular condition. The gene signature aids in determining a group of subjects at a specific state of a disease and provides suggestions on treatments.

### Content Management System

A CMS of the disclosure can comprise:
I) an information input module;
II) a report assignment module;
III) a review module;
IV) an output module; and
V) a report update module.
   In some embodiments, the information input module receives information obtained from the genetic analysis of a biological sample (genetic analysis information). Non-limiting examples of biological samples include tissue, tumor samples, cells, blood, semen, saliva, cell-free DNA, and cell-free RNA. Upon receipt of the information obtained from the genetic analysis by the information input module, the genetic analysis information can be analyzed by the report assignment module to generate an assignment of a LLR to the subject. The initial assignment can be a tentative assignment or a final assignment. In instances when a tentative assignment is generated, the tentative assignment can be stored by the review module until it is reviewed by an administrator or super administrator. Once reviewed, the tentative assignment can be approved by an administrator or super administrator to thereby generate an assigned LLR.

LLRs can comprise informational text pertaining to information obtained from a genetic analysis. In some embodiments, the information text of a LLR is written by one or more certified genetic counselors and/or vetted by patient advocates. The information text can contain information related to genetic counseling. A LLR can be assigned to a subject based on individual gene mutations. A LLR can be translated into two streams, one comprising informational text for a healthcare provider and one comprising information text for a patient. In some embodiments, the information texts are updated based on information learned from new studies. In some embodiments the informational text is organized in question and answer form. Question and answer combinations can be tagged across multiple specialties (e.g. cancer genetics, cardiovascular genetics) within the CMS. Content and language used in the informational text can be tailored to end user type. For example, the patient questions and answers can use lay language and other portions of the informational text can provide information that would benefit and interest a patient.

The question and answer content housed in the CMS can be subject to logging, role-based security, and/or reporting. For example, a super administrator's account has access to the entire database of questions and answers within the CMS and the corresponding DDM it is built on. The super administrators can grant access to sections of the database to other administrators. This access grant allows administrators to create and update the question and answer content but not push updates live until reviewed by a super administrator.

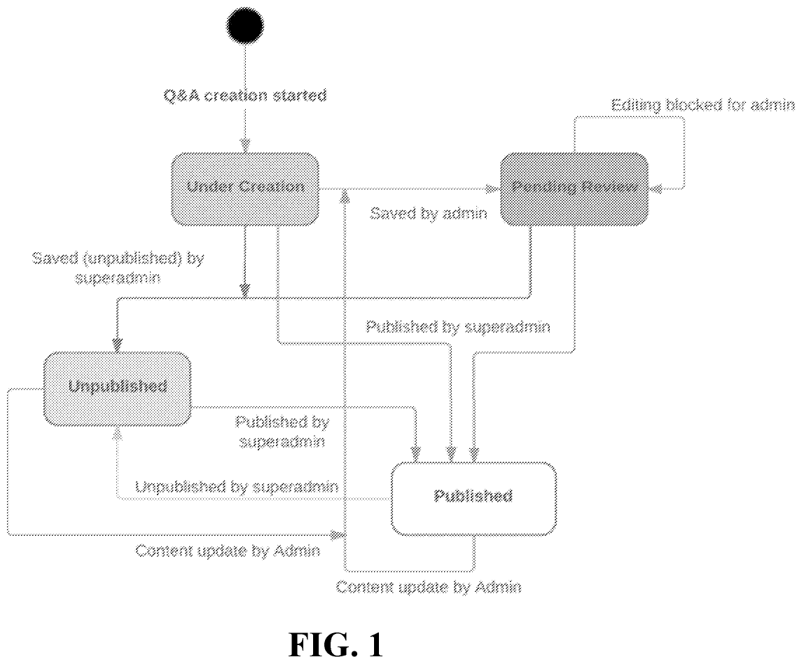

FIG. 1 illustrates a process for editing the questions and answers and granting of permission rights. First, the questions and answers combinations are generated. In some embodiments, the question and answer combinations are saved by the administrator for pending review or saved as unpublished by the super administrator. In some embodiments, the question and answer combinations are published by the super administrator. Following the pending review, the combination is published or saved as unpublished by the super administrator. Alternatively, the editing following review can be blocked by the administrator. Although the combination can be saved as unpublished, the super administrator can publish the combination, or the administrator may save the content for pending review. Following publication, the question and answer content can still be updated by the administrator and saved for pending review. Alternatively, the super administrator can remove the content from publication.

Non-limiting examples of topics that can be contained in the informational text of an LLR include information on genes found to contain a variant, explanation of variant effect on gene function, interpretation of medical consequences of genetic variant (e.g. cancer risk, risk of cardiovascular disease), medical management insights based on national guidelines and published literature, information on implications for family members, clinical trials for which a user may qualify, and/or educational support and clinical resources. The educational support and clinical resources are, for example, patient advocacy groups or medical specialist types for consideration of consultation. In some embodiments, the patient receives invitations to research studies while protecting the health information of the patient.

LLRs can comprise informational text that presents information pertaining to a phenotype associated with a genetic variant. Non-limiting examples of phenotypes an informational text can pertain to include hereditary cancer syndromes such as breast cancer, ovarian cancer, fallopian tube cancer, pancreatic cancer, prostate cancer, melanoma, uterine cancer, colon cancer, gastric cancer, thyroid cancer, and kidney cancer; and hereditary cardiac syndromes such as familial hypercholesterolemia, cardiomyopathy, aortic disease and arrhythmic disorders.

Phenotypes covered by a LLR can be associated with a mutation in any gene. Non-limiting examples of genes that can have a mutations associated with a phenotype that is covered in a LLR include BRCA1, BRCA2, APC, ATM, BARD1, BMPR1A, BRIP1, CDH1, CDK4, CDKN2A, CHEK2, EPCAM, GREM1, HOXB13, MEN1, MLH1, MSH2, MSH2-EPCAM, MSH6, NBN, NF2, PALB2, PMS2, POLD1, POLE, PTEN, RAD51C, RAD51D, RB1, RET, SDHAF2, SDHB, SDHC, SDHD, SMAD4, STK11, TP53, TSC1, TSC2, VHL, WT1, LDLR, APOB, PCSK9, ACTC1, GLA, LMNA, MYBPC3, MYH7, MYL2, MYL3, PRKAG2, TNNI3, TNNT2, TPM1, ACTA2, FBN1, MYH11, SMAD3, TGFBR1, TGFBR2, COL3A1, DSP, DSC2, TMEM43, DSG2, PKP2, KCNQ1, KCNH2, SCN5A, and RYR2.

In some embodiments, the CMS further comprises a term bank module that includes banks of terms with definitions that end users can explore further when accessing LLRs. In some embodiments a user explores a term by hovering a mouse icon over the term (i.e. a hover function).

In some embodiments, the LLRs are created within the CMS through the DDM. The DDM can be an algorithm that determines the hierarchical order and structure of data when connecting an existing genetic testing lab report with the database content. Several hard-coded rules can provide a basic structure, while other rules can be altered by the super administrators to allow for flexibility and adaptability of the system.

A LLR can include two information streams dependent on the end user. Each information stream can comprise a different informational text. One of the streams is, for example, for clinicians who have ordered testing for their patients. The other stream is, for example, for patients and users who are direct customers keying in and uploading test results. Both streams of information can come from the question and answer database content. The DDM can allow for tagging of such content, which aids in the creation of LLRs and navigating the database. In some embodiments, the LLR is displayed by an output module. In some embodiments the output module can display the LLR through a healthcare provider (e.g. clinician) portal or a patient portal. In some embodiments the healthcare provider portal parallels the patient portal. The clinician portal can also include a direct view into the patient's portal so the clinician and patient are viewing the same questions and answers. The streams can be evidence-based and fully referenced streams.

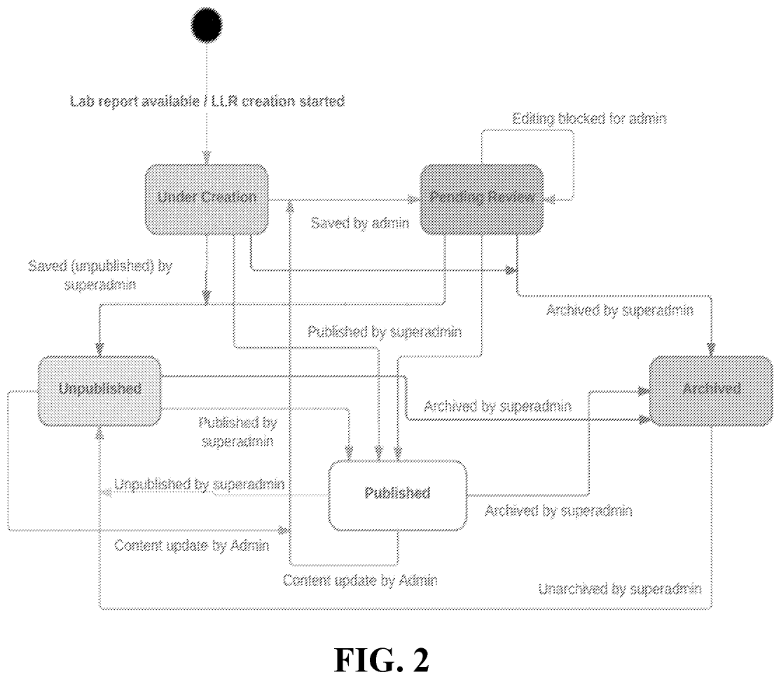

FIG. 2 illustrates an illustrative process for editing of the LLRs and granting of permission rights. First, the LLR is generated. In some embodiments, the LLRs are saved as unpublished by the administrator or super administrator, or are saved by the administrator for pending review. In some embodiments, the LLRs are archived by the super administrator or published by the super administrator. Following review, the LLRs are saved as unpublished or archived by the super administrator. In some embodiments, the LLRs are published by the super administrator or have the editing blocked by the administrator. Once archived, the LLRs are removed from the archive by the super administrator. In some embodiments, the unpublished LLRs have content updated by the administrator or published by the super administrator. Alternatively, the unpublished LLRs are archived by the super administrator. Once published, the LLR content is updated by the administrator and saved for pending review. Alternatively, the published LLR is removed from publication or the published LLR is archived by the super administrator.

In some embodiments, the LLRs are linked to the genetic analysis in the report assignment module. When a direct user or testing lab partner provides a genetic test result with the information input module, the DDM is utilized to match the request with a corresponding LLR from the CMS. The LLR is then displayed to the user within the output module. Each data input can be considered in a hierarchical manner by the DDM to correctly perform this linkage.

An administrator can match a test result with the appropriate LLR. Before publication, the match can be approved by a super administrator. Approval can be required for initial LLR matching and for matching new LLRs to a genetic test result or user, based on new information. The matching of the test result and LLR can be automated.

Non-limiting examples of hierarchical data inputs include test type, subspecialty, result type, the gene in which the variant is detected, and the variant name. The test type can be a classification of the kind of genetic test utilized. The genetic test can be somatic, e.g. genetic testing of tumor tissue, or germline, e.g., testing of DNA endogenous to the person. The subspecialty can be, for example, ancestry, cancer, or cardiovascular. The result type can be a negative result, a disease-causing variant or a variant of uncertain significance. The gene name can be the name of the gene in which the genetic change was detected, for example, BRCA1, MLH1, APOB or ACTA2. The variant name can be the name of the genetic alteration or difference detected if the test result was not negative.

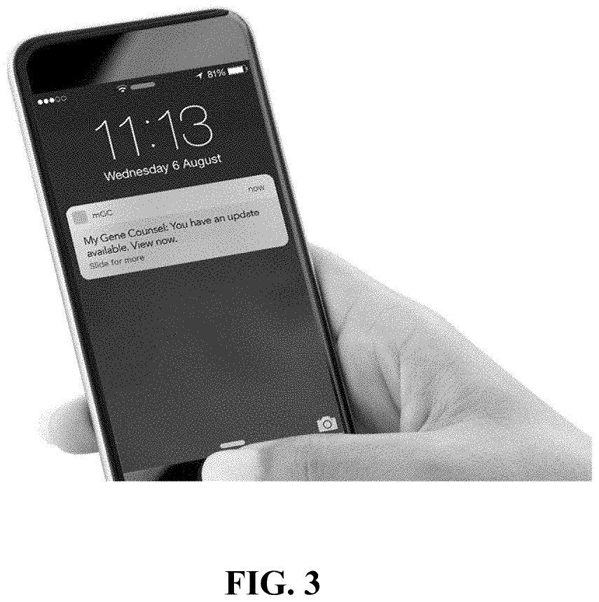

While the hierarchical data inputs allow for the building of tailored LLRs to link with each test, the flexibility of the CMS can allow the LLRs to be dynamic such that the information streams comprising informational text can be updated via a report update module as new data emerge. When the informational text of a LLR is updated based on new data, the output module can release changes to every user to whom the update applies. For example, if a new medical recommendation occurs for those with a disease-causing variant in a specific gene, the specific gene's informational text (e.g. the questions and answers) for medical management are updated. If one of the questions and answers is present in 3,000 LLRs, the 3,000 LLRs can be instantaneously updated. The direct administrator portal can then take those 3,000 updated LLRs and link the LLRs with any associated subjects or users. For example, if each of the 3,000 LLRs is linked with 10 users, then 30,000 total users are linked. Those 30,000 direct users can be notified by a communication module contained within the CMS that can notify users by, for example, e-mail or text message. Users can view the latest information via the output module. FIG. 3 illustrates an example push notification alerting user to the LLR update. The push notifications can comprise information on how variants are reclassified, clinical information updates, or when clinical trial opportunities become available.

Non-limiting examples of updates to LLR and potential user notification include reclassification of genetic variants, the effect of which was previously uncertain, updates to medical management guidelines in association with genetic variants, and/or new clinical trials for which the patient or user may qualify. For the reclassification of genetic variants, the effect of which was previously uncertain, an example notification is a variant of uncertain significance reclassified to disease-causing or benign.

### Output Module

In some embodiments the output module comprises a direct web portal. The direct web portal can be the consumer-facing web portal that serves to provide relevant LLR details to end users. The portal can rely on the CMS for fetching DDM and LLR data, which the direct web portal then displays. The direct web administrator can also allow backend work to be performed by the administrators. In some embodiments, the direct web portal includes consumer-facing elements and example features such as user registration features, plan selection features, user sign-in features, features to place and view LLR requests, features to view LLRs, and profile maintenance features. In some embodiments, the portal can include administrator-facing elements and example features such as administrator sign-in features, features to view or search for LLR requests, features to check the status of LLR requests (e.g. pending, under review, LLR available or not available), features for the management of LLR requests (e.g. publish LLR, delete LLR), features for user management (e.g. re-send password or send email notification), and features to view purchases by users, manage organizations (e.g. lab partners), view user feedback, and manage administrators (e.g. assign subspecialties such as cancer or cardiac disorders).

In some embodiments, the direct web portal may partner with genetic testing laboratories, genomic population studies, pharmaceutical companies, referral services, or database and invitation services. The direct web portal may be a secure, scalable, unbiased method through which genomic study partners can receive their genomic data digitally. In some embodiments, pharmaceutical companies may conduct broad genetic testing on clinical trial participants to enable identification of new drug targets. These companies may use the direct web portal for the return of genetic test results.

### User and Third-Party Interaction

In some embodiments, information providers such as clinicians and genetic testing laboratories can directly interact with a CMS disclosed herein. Information providers can upload genetic analyses and information obtained from genetic analyses directly to an information storage module contained within the content managements system. Stored information can then be retrieved by the information input module.

In some embodiments, the output module can display an informational text to the user that comprises a question. The information input module can receive an answer to the question from the user via, for example, the direct web portal. The answer can be used to, for example, inform the assignment report assignment module in generating an assignment or to provide information to third-parties that can be used for research purposes.

### Computer Control Systems

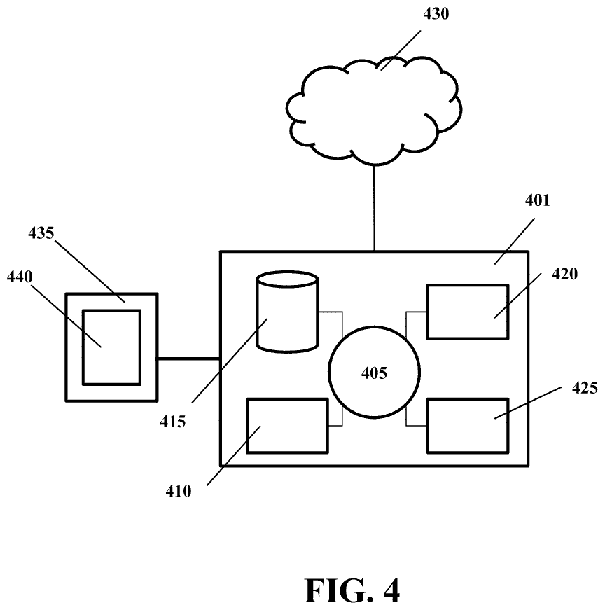

The present disclosure provides computer control systems that are programmed to implement methods of the disclosure. FIG. 4 shows a computer system 401 that is programmed or otherwise configured to implement the methods of the present disclosure. The computer system 401 can regulate various aspects of web-based content management system. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user (e.g., an operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440. The UI can allow a user to set various conditions for the methods described herein, for example, determining the hierarchical order and structure of the LLR. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example, determine the hierarchical order and structure of the LLR.

### EXAMPLES

The examples below are illustrative and non-limiting.

### Example 1

The LLR is created using DDM and CMS for a patient who underwent medical grade testing and found to have a disease-causing genetic variant (mutation) in the BRCA1 gene. The user inputs mygenecounsel.com into a web browser. The user can then create an account and upload or enter detail on a genetic test result. The DDM and CMS define the user's request and match the user request with the appropriate LLR, which includes applicable genetic counseling information. The administrator can review the request or matched results and can publish the LLR for the user to view. The user is notified that his or her LLR is available. The user is updated with notifications as important changes occur to the LLR by e-mail or short message service (SMS).

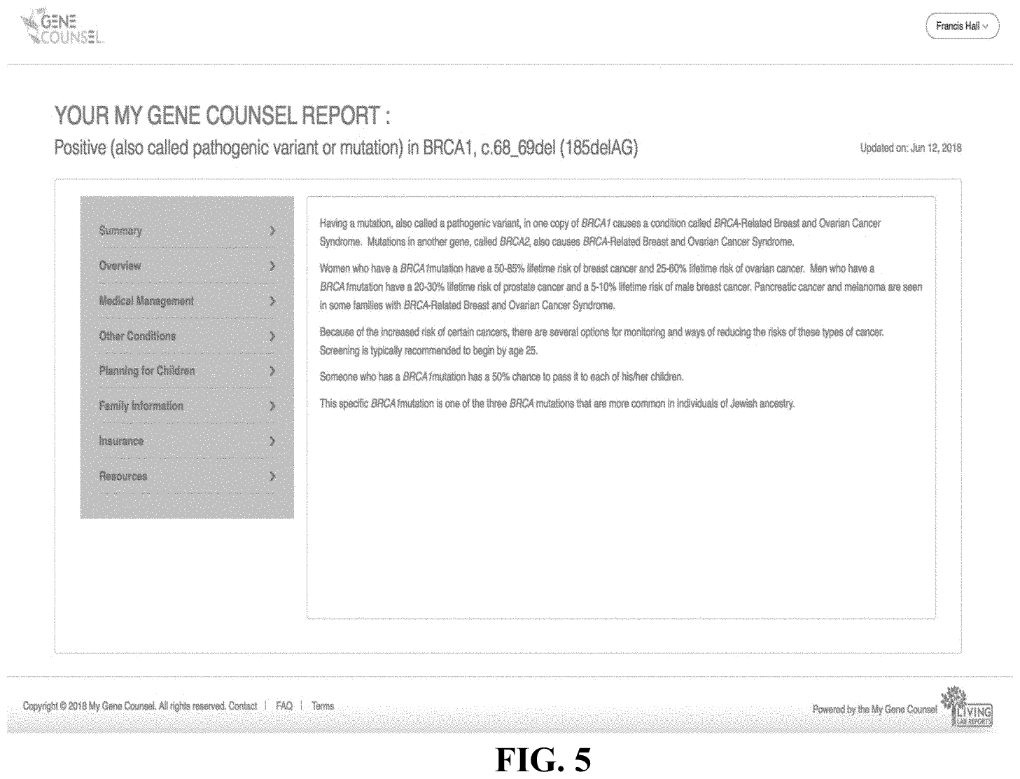

FIG. 5 illustrates an interactive browser displaying an LLR view on the web portal comprising a list of options that the user can access. In this instance, the user tested positive for a pathogenic variant or mutation in BRCA1. The left panel of the LLR provides options for the user to view the summary, overview, medical management, other conditions, planning for children, family information, insurance, and other resources relating to their test result. When the user selects the summary icon, the user sees the following description.

Having a mutation, also called a pathogenic variant, in one copy of BRCA1 causes a condition called BRCA-Related Breast and Ovarian Cancer Syndrome. Mutations in another gene, called BRCA2, also causes BRCA-Related Breast and Ovarian Cancer Syndrome. Women who have a BRCA1 mutation have a 50-85% lifetime risk of breast cancer and 25-60% lifetime risk of ovarian cancer. Men who have a BRCA1 mutation have a 20-30% lifetime risk of prostate cancer and a 5-10% lifetime risk of male breast cancer. Pancreatic cancer and melanoma are seen in some families with BRCA-Related Breast and Ovarian Cancer Syndrome. Because of the increased risk of certain cancers, several options exist for monitoring and reducing the risks of these types of cancer. Screening is typically recommended to begin by age 25. Someone who has a BRCA1 mutation has a 50% chance to pass the mutation to children. The specific BRCA1 mutation is one of the three BRCA mutations that are more common in individuals of Jewish ancestry.

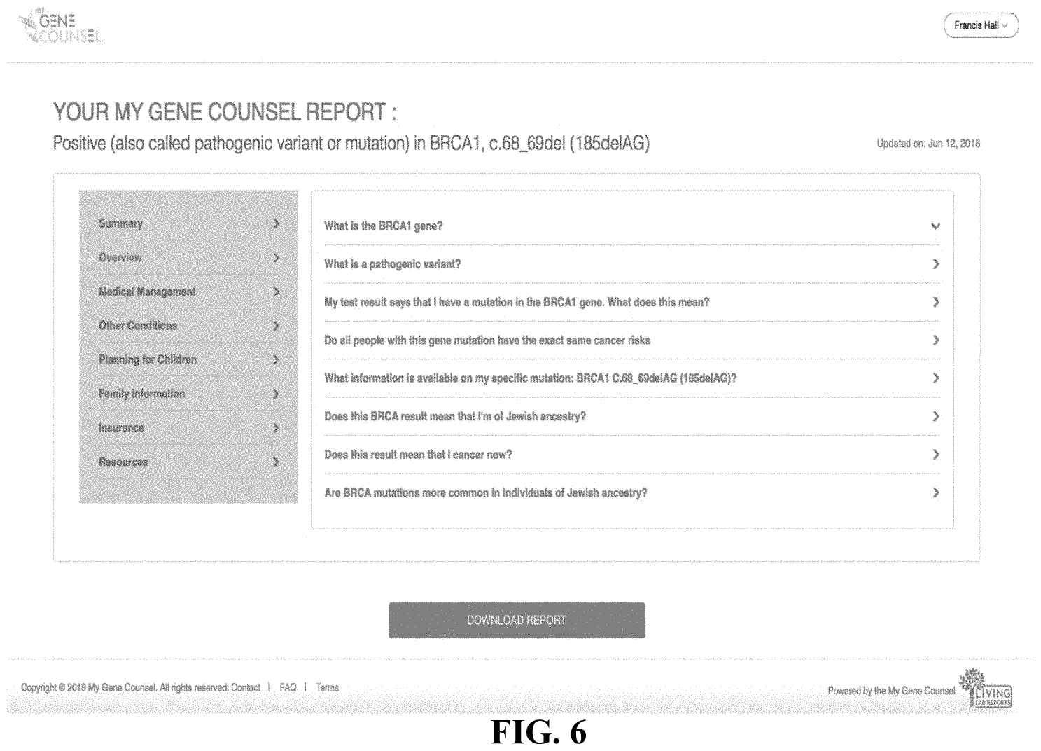

FIG. 6 illustrates an interactive browser expanding the overview tab of the LLR assigned to a user that tested positive with a pathogenic variant or mutation in BRCA1. Upon expansion of the overview icon, a list of questions appears. The questions are directed to detailing what a BRCA1 gene is, what a pathogenic variant is, the meaning of a mutation in the BRCA1 gene, whether all people with the gene mutation have the same cancer risks, available information on the specific mutation, whether the BRCA result indicates if one is of Jewish ancestry, whether the result means that the individual has cancer, and whether BRCA mutations are more common in individuals of Jewish ancestry. The web page also has an option of downloading the report.

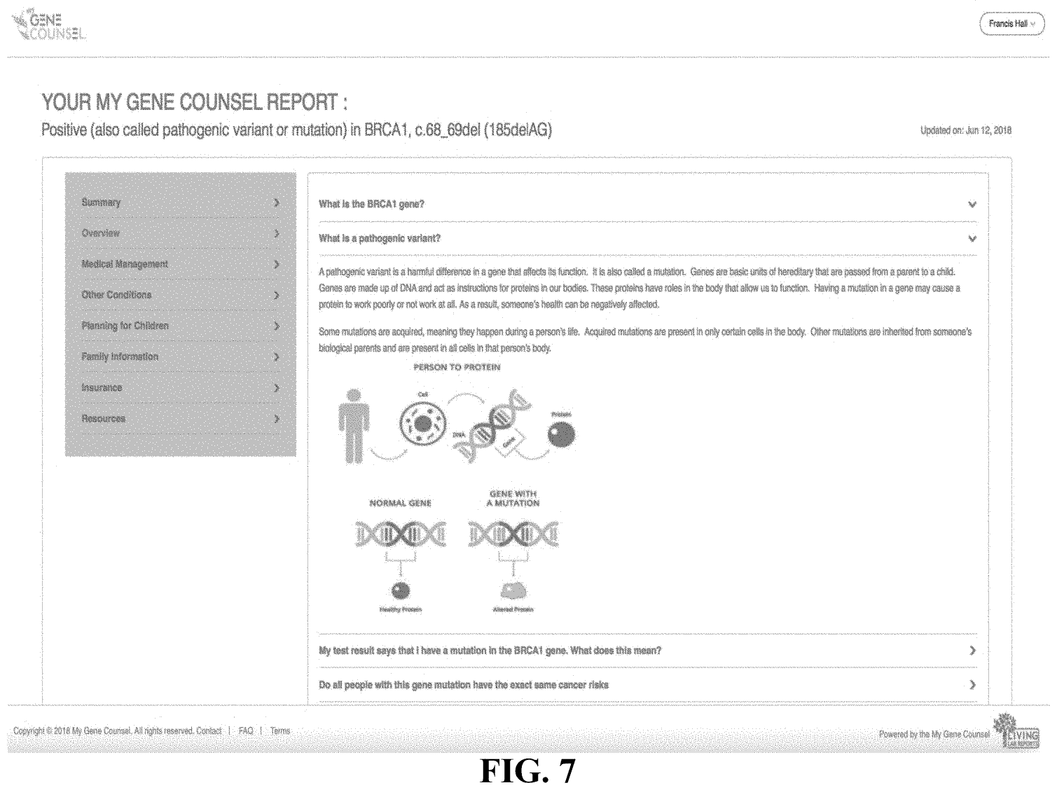

FIG. 7 illustrates the screen view of FIG. 6 once the user expands the question asking what a pathogenic variant is. The answer states that a pathogenic variant is a harmful difference in a gene that affects gene function. The variant is also called a mutation, as illustrated in the figure. Genes are basic units of hereditary that are passed from a parent to a child. Genes are made of DNA and act as instructions for proteins in our bodies. These proteins have roles in the body that allow us to function. Such a description is also illustrated in the figure. Having a mutation in a gene can cause a protein to work poorly or not work at all. As a result, someone's health can be negatively affected. Some mutations are acquired, meaning that the mutation happens during a person's life. Acquired mutations are present in only certain cells in the body. Other mutations are inherited from someone's biological parents and are present in all cells in that person's body.

### Example 2

Labs can partner with the direct portal through direct integration using an application programming interface (API). A patient undergoes genetic testing through a physician, and that physician uses the partnered lab. Through the partnership, that customer (patient and/or clinician) has access to the web portal and thus a corresponding LLR.

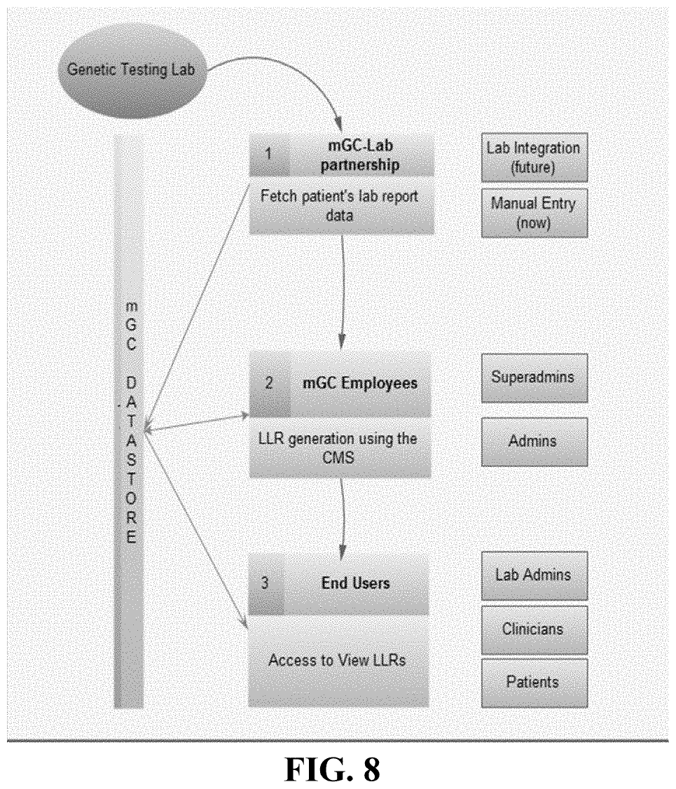

FIG. 8 illustrates a lab integration data flow diagram. The user undergoes genetic testing through a doctor. The doctor can use a lab from the partnered list. When the user's genetic test is complete, the system can fetch the resulting data using API and send the data to the data store. Such a process can either involve lab integration or a manual entry. The administrator or super administrator can review the result, match the result with the appropriate LLR, and publish the LLR for the user to view. The LLR can be generated using the CMS and access can be given to the end users. The end users can be lab administrators, clinicians, or patients. Alternatively, the LLRs can be generated directly from the data store and access can be granted to the end users. The user can be notified that the LLR is available. The user can be updated with notifications as important changes occur to the LLR by e-mail or SMS.

### EMBODIMENTS

The following non-limiting embodiments provide illustrative examples of the methods, products, and systems disclosed herein, but do not limit the scope of such methods, products and systems.

### Embodiment 1

A computer program product comprising a non-transitory computer-readable medium having computer-executable code encoded therein, the computer-executable code adapted to be executed to implement a method comprising: a) providing a content management system, wherein the content management system comprises: I) an information input module; II) a report assignment module; III) a review module; IV) an output module; and V) a report update module; b) receiving, by the information input module, information obtained from a genetic analysis of a biological sample from a subject; c) performing an assignment analysis, by the report assignment module, of the information obtained from the genetic analysis; d) generating, by the report assignment module, a tentative assignment of a report to the subject based on the assignment analysis, wherein the report comprises an informational text that pertains to the information obtained from the genetic analysis; e) storing, by the review module, the tentative assignment, wherein the tentative assignment remains stored until the tentative assignment is reviewed and approved by an administrator; f) generating by the review module, based on receipt of approval by the administrator, an assigned report comprising the informational text; g) transmitting the assigned report from the review module to the output module; h) displaying by the output module the assigned report to a user; i) updating, by the report update module, the informational text, thereby generating an updated report, wherein the updated report is generated after a new piece of information is received by the report update module; and j) transmitting the updated report from the report update module to the output module, wherein the output module displays the updated report to the user.

### Embodiment 2

The computer program product of embodiment 1, wherein: i) the content management system further comprises a communication module; and ii) the method further comprises: a) generating by the communication module a message to the user, wherein the message informs the user that the updated report is available for viewing; and b) transmitting by the communication module the message to the user.

### Embodiment 3

The computer program product of embodiment 1 or 2, wherein the information obtained from the genetic analysis is genome sequencing data.

### Embodiment 4

The computer program product of any one of embodiments 1-3, wherein the informational text presents information pertaining to a phenotype associated with a genetic variant.

### Embodiment 5

The computer program product of any one of embodiments 1-4, wherein the new piece of information pertains to a genotype of the subject.

### Embodiment 6

The computer program product of any one of embodiments 1-4, wherein the new piece of information pertains to a phenotype of the subject.

### Embodiment 7

The computer program product of any one of embodiments 1-6, wherein: i) the content management system further comprises an information storage module; and ii) (b) comprises: A) receiving by the information storage module the information obtained from the genetic analysis from an information provider; and B) retrieving by the information input module the information obtained from the genetic analysis from the information storage module.

### Embodiment 8

The computer program product of any one of embodiments 1-7, wherein the informational text comprises a message regarding a clinical trial.

### Embodiment 9

The computer program product of any one of embodiments 1-8, wherein: the informational text comprises a question; and b) the method further comprises receiving by the information input module an answer to the question from the user.

What is claimed is:

1. A computer program product comprising a non-transitory computer-readable medium having computer-executable code encoded therein, the computer-executable code adapted to be executed to implement a method comprising:
a) providing a content management system, wherein the content management system comprises:
I) an information input module;
II) a report assignment module;
III) a review module;
IV) an output module; and
V) a report update module;
b) receiving, by the information input module, information obtained from a genetic analysis of a biological sample from a subject;
c) performing an assignment analysis, by the report assignment module, of the information obtained from the genetic analysis;
d) generating, by the report assignment module, a tentative assignment of a report to the subject based on the assignment analysis, wherein the report comprises an informational text that pertains to the information obtained from the genetic analysis;
e) storing, by the review module, the tentative assignment, wherein the tentative assignment remains stored until the tentative assignment is reviewed and approved by an administrator;
f) generating by the review module, based on receipt of approval by the administrator, an assigned report comprising the informational text;
g) transmitting the assigned report from the review module to the output module;
h) displaying by the output module the assigned report to a user;
i) updating, by the report update module, the informational text, thereby generating an updated report, wherein the updated report is generated after a new piece of information is received by the report update module; and
j) transmitting the updated report from the report update module to the output module, wherein the output module displays the updated report to the user.

2. The computer program product of claim 1, wherein:
i) the content management system further comprises a communication module; and
ii) the method further comprises:
a) generating by the communication module a message to the user, wherein the message informs the user that the updated report is available for viewing; and
b) transmitting by the communication module the message to the user.

3. The computer program product of claim 1, wherein the information obtained from the genetic analysis is genome sequencing data.

4. The computer program product of claim 1, wherein the informational text presents information pertaining to a phenotype associated with a genetic variant.

5. The computer program product of claim 1, wherein the new piece of information pertains to a genotype of the subject.

6. The computer program product of claim 1, wherein the new piece of information pertains to a phenotype of the subject.

7. The computer program product of claim 1, wherein:
i) the content management system further comprises an information storage module; and
ii) (b) comprises:
A) receiving by the information storage module the information obtained from the genetic analysis from an information provider; and
B) retrieving by the information input module the information obtained from the genetic analysis from the information storage module.

8. The computer program product of claim 1, wherein the informational text comprises a message regarding a clinical trial.

9. The computer program product of claim 1, wherein:
a) the informational text comprises a question; and
b) the method further comprises receiving by the information input module an answer to the question from the user.
